(19) United States
(12) Patent Application Publication
(10) Pub. No.: US 2024/0420805 A1
(43) Pub. Date: Dec. 19, 2024

(54) INFORMATION PROCESSING SYSTEM, INFORMATION PROCESSING METHOD, AND INFORMATION PROCESSING PROGRAM

(71) Applicant: Resonac Corporation, Minato-ku, Tokyo (JP)

(72) Inventor: Minoru HOSHINO, Minato-ku, Tokyo (JP)

(21) Appl. No.: 18/704,220

(22) PCT Filed: Oct. 27, 2022
(86) PCT No.: PCT/JP2022/040239

§ 371 Date: Jun. 7, 2024

(30) Foreign Application Priority Data
Nov. 2, 2021 (JP) 2021-179277

(51) Int. Cl. G16C 20/10

(52) U.S. Cl. CPC G16C 20/10

(57) ABSTRACT

An information processing system according to an example includes at least one processor. The at least one processor acquires a reactant list indicating a plurality of reactants, acquires a reaction formula expressing a reactant having a reactive functional group by a general formula, selects at least one combination of the reactants compatible with the reaction formula as at least one reactant combination from the reactant list, and identifies, for each of the at least one reactant combination, a product to be obtained from the reactant combination by the reaction formula.

### TECHNICAL FIELD

One aspect of the present disclosure relates to an information processing system, an information processing method, and an information processing program.

### BACKGROUND ART

A method for searching a synthesizable compound is known. For example, Patent Literature 1 describes a chemical reaction transition state search system for obtaining a chemical structure of a target transition state in a chemical reaction.

### CITATION LIST

### Patent Literature

Patent Literature 1: Japanese Unexamined Patent Publication No. 2010-97371

### SUMMARY OF INVENTION

### Technical Problem

A method for efficiently searching a synthesizable compound is desired.

### Solution to Problem

An information processing system according to one aspect of the present disclosure includes at least one processor. The at least one processor is configured to: acquire a reactant list indicating a plurality of reactants; acquire a reaction formula expressing a reactant having a reactive functional group by a general formula; select at least one combination of the reactants compatible with the reaction formula as at least one reactant combination from the reactant list; and identify, for each of the at least one reactant combination, a product to be obtained from the reactant combination by the reaction formula.

An information processing method according to one aspect of the present disclosure is executed by an information processing system including at least one processor. The information processing method includes: acquiring a reactant list indicating a plurality of reactants; acquiring a reaction formula expressing a reactant having a reactive functional group by a general formula; selecting at least one combination of the reactants compatible with the reaction formula as at least one reactant combination from the reactant list; and identifying, for each of the at least one reactant combination, a product to be obtained from the reactant combination by the reaction formula.

An information processing program according to one aspect of the present disclosure causes a computer to execute: acquiring a reactant list indicating a plurality of reactants; acquiring a reaction formula expressing a reactant having a reactive functional group by a general formula; selecting at least one combination of the reactants compatible with the reaction formula as at least one reactant combination from the reactant list; and identifying, for each of the at least one reactant combination, a product to be obtained from the reactant combination by the reaction formula.

In such aspects, by selecting the reactant combination and identifying the product based on the reaction formula focusing on the reactive functional group that directly contributes to a chemical reaction, it is possible to efficiently search a synthesizable compound.

### Advantageous Effects of Invention

According to one aspect of the present disclosure, it is possible to efficiently search the synthesizable compound.

### BRIEF DESCRIPTION OF DRAWINGS

FIG. 1 is a diagram illustrating an example of a functional configuration of an information processing system.

FIG. 2 is a diagram illustrating an example of a hardware configuration of a computer configuring the information processing system.

FIG. 3 is a flowchart illustrating an example of processing in the information processing system.

FIG. 4 is a diagram illustrating an example of reactants and reaction formulae.

FIG. 5 is a diagram illustrating an example of reaction formula lists.

FIG. 6 is a diagram illustrating an example of processing for one reaction formula list.

FIG. 7 is a diagram illustrating chemical reactions and products to be obtained by the processing illustrated in FIG. 6.

FIG. 8 is a diagram illustrating an example of processing for another reaction formula list.

FIG. 9 is a diagram illustrating chemical reactions and products to be obtained by the processing illustrated in FIG. 8.

FIG. 10 is a diagram illustrating another example of the functional configuration of the information processing system.

FIG. 11 is a flowchart illustrating another example of the processing in the information processing system.

FIG. 12 is a diagram illustrating an example of extracting a target atom.

### DESCRIPTION OF EMBODIMENTS

Hereinafter, an embodiment in the present disclosure will be described in more detail with reference to the attached drawings. In the description of the drawings, the same reference numerals will be applied to the same or equivalent elements, and the repeated description will be omitted.

### [Configuration of System]

An information processing system 10 according to an embodiment is a computer system for searching a synthesizable compound. For example, the information processing system 10 searches a synthesizable organic compound. The information processing system 10 acquires a reaction formula expressing reactants having a reactive functional group by a general formula, and selects a combination of the reactants compatible with the reaction formula as a reactant combination. Then, the information processing system 10 identifies a product to be obtained from the reaction formula and the reactant combination. The product is a compound estimated to be synthesizable. The information processing system 10 searches realizable chemical reaction and compound from a group of given reaction formulae indicating a reactive functional group and a group of given reactants. For example, the group of the reaction formulae is a set of realizable reaction formulae, and the group of the reactants is a set of available reactants. The information processing system 10 is expected to contribute to materials informatics in which a new or useful compound is efficiently searched via information science.

The reactive functional group refers to an atom group relevant to the generation or cleavage of a bond in a chemical reaction. In the present disclosure, the “reaction formula expressing the reactant having a reactive functional group by the general formula” refers to a reaction formula in which part of the reactant other than the reactive functional group is expressed by a placeholder symbol such as R, R₁, and R₂.

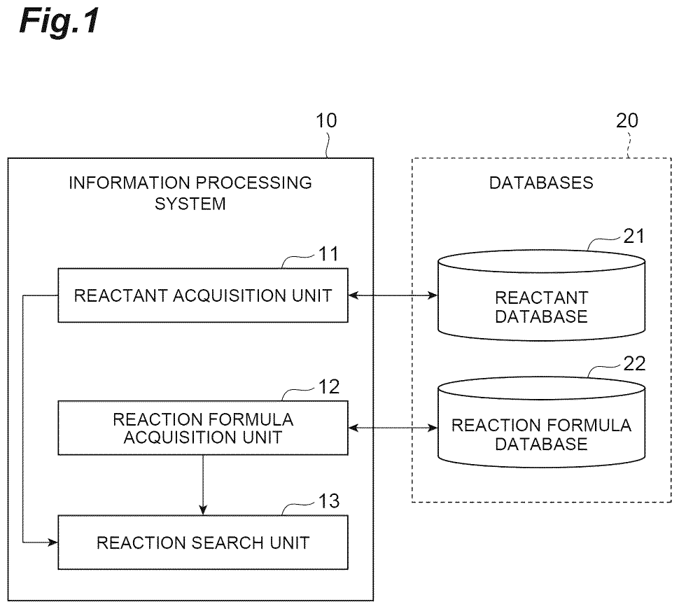

FIG. 1 is a diagram illustrating an example of a functional configuration of the information processing system 10. The information processing system 10 includes a reactant acquisition unit 11, a reaction formula acquisition unit 12, and a reaction search unit 13, as function modules. The reactant acquisition unit 11 is a function module acquiring a reactant list indicating a plurality of reactants. The reaction formula acquisition unit 12 is a function module acquiring a reaction formula list indicating at least one reaction formula expressing a reactant having a reactive functional group by a general formula. The reaction search unit 13 is a function module selecting a reactant combination compatible with the acquired reaction formula from the reactant list and identifying a product to be obtained from the reactant combination by the reaction formula.

In the present disclosure, a “combination of the reactants compatible with the reaction formula” and the “reactant combination compatible with the reaction formula” refer to a combination of the reactants having a reactive functional group indicated by the reaction formula. The reactant combination is a combination of at least two reactants. For example, assuming that general formulae of two reactants indicated by the left member of the reaction formula are referred to as a first general formula and a second general formula, the “combination of the reactants compatible with the reaction formula” and the “reactant combination compatible with the reaction formula” are a combination of a reactant having a reactive functional group of the first general formula and a reactant having a reactive functional group of the second general formula. In this example, in a case where the reactive functional groups are the same in the first general formula and the second general formula, the reactant combination may be generated by the same two reactants.

In an example, the information processing system 10 is connected to databases 20 via a given communication network. The communication network may be configured by including at least one of the Internet and an intranet. The communication network may be configured by using at least one of a wired network and a wireless network. The databases 20 is a set of databases that is a non-transitory storage device storing data used by the information processing system 10. In an example, the databases 20 includes a reactant database 21 and a reaction formula database 22.

The reactant database 21 is a database storing reactant data indicating each of the reactants. In an example, the reactant data indicates available reagents.

The reaction formula database 22 is a database storing reaction formula data indicating each of the reaction formulae. As described above, each of the reaction formulae expresses the reactants having a reactive functional group by the general formula. In an example, the reaction formula data indicates at least one of a realizable reaction formula, an easy-to-handle reaction formula, and a reaction formula of which the yield is a given threshold or more.

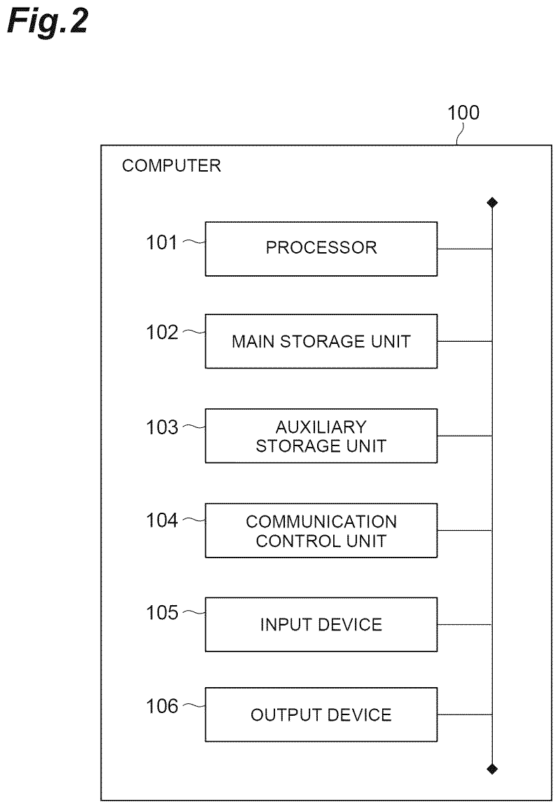

FIG. 2 is a diagram illustrating an example of a general hardware configuration of a computer 100 configuring the information processing system 10. For example, the computer 100 includes a processor 101 such as CPU, which executes an operating system, an application program, and the like, a main storage unit 102 composed of ROM and RAM, an auxiliary storage unit 103 composed of a hard disk, a flash memory, and the like, a communication control unit 104 composed of a network card or a wireless communication module, an input device 105 such as a keyboard and a mouse, and an output device 106 such as a monitor.

Each function module of the information processing system 10 is realized by reading a predetermined program on the processor 101 or the main storage unit 102 and causing the processor 101 to execute the program. The processor 101 operates the communication control unit 104, the input device 105, or the output device 106 in accordance with the program, and performs the reading and the writing of the data in the main storage unit 102 or the auxiliary storage unit 103. The data or the database required for processing is stored in the main storage unit 102 or the auxiliary storage unit 103.

The information processing system 10 is composed of one or more computers. In a case of using a plurality of computers, these computers are connected via a communication network such as the Internet and an intranet to construct one information processing system 10 logically.

An information processing program for causing a computer to function as the information processing system 10 includes program code for realizing each function module of the information processing system 10. The information processing program may be provided after being non-transitorily recorded in a tangible recording medium such as CD-ROM, DVD-ROM, and a semiconductor memory. Alternatively, the information processing program may be provided as a data signal superimposed on a carrier wave via a communication network. The provided information processing program is stored in the auxiliary storage unit 103, for example.

### [Operation of System]

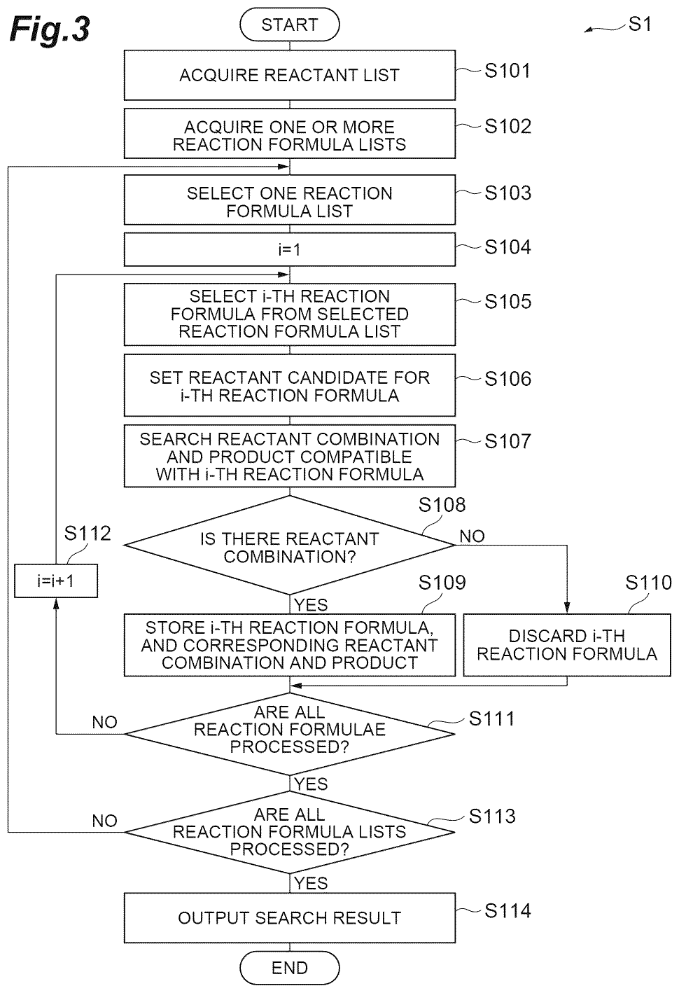

With reference to FIG. 3, an operation of the information processing system 10 will be described, and an information processing method according to this embodiment will be described. FIG. 3 is a flowchart illustrating an example of processing in the information processing system 10 as a processing flow S1.

In step S101, the reactant acquisition unit 11 acquires the reactant list. In an example, the reactant acquisition unit 11 accesses the reactant database 21 to read out the reactant data indicating a plurality of reactants. The reactant acquisition unit 11 acquires a set of the reactants as the reactant list.

In step S102, the reaction formula acquisition unit 12 acquires one or more reaction formula lists. In an example, the reaction formula acquisition unit 12 accesses the reaction formula database 22 to read out the reaction formula data indicating one or more reaction formulae. Subsequently, the reaction formula acquisition unit 12 generates each of a plurality of arrangement patterns for one or more reaction formulae as the reaction formula list. In an example, the number of reaction formula lists to be generated is the total number ₙPᵣ of permutations that may be generated using r different reaction formulae among n different reaction formulae. Since r=n, the total number of permutations is the factorial of a positive number n. For example, in a case where four reaction formulae are obtained, the reaction formula acquisition unit 12 generates 24 reaction formula lists each indicating the arrangement of 4 reaction formulae. The reaction formula acquisition unit 12 may set the order of reaction formulae by another method. For example, the reaction formula acquisition unit 12 may generate the reaction formula list in accordance with the order of the reaction formulae designated by a user. Alternatively, the reaction formula acquisition unit 12 may generate the reaction formula list based on a given order relevant to the reactive functional group. In a case where the reaction formula acquisition unit 12 reads out one reaction formula from the reaction formula database 22, a single reaction formula list to be generated indicates a single reaction formula.

In step S103, the reaction search unit 13 selects one reaction formula list.

In step S104, the reaction search unit 13 sets a variable i for selecting one reaction formula from the reaction formula list to 1. The variable i represents the order of the reaction formulae in one reaction formula list.

In step S105, the reaction search unit 13 selects the i-th reaction formula from the selected reaction formula list.

In step S106, the reaction search unit 13 sets a reactant candidate for the i-th reaction formula. For the first reaction formula, the reaction search unit 13 sets the entire reactant list as reactant candidates. A method for setting the reactant candidates for the second and subsequent reaction formulae will be described below.

In step S107, the reaction search unit 13 searches a reactant combination and a product compatible with the i-th reaction formula. The reaction search unit 13 selects the reactant combination compatible with the i-th reaction formula from the reactant candidates. Then, the reaction search unit 13 identifies a product to be obtained from the reactant combination by the reaction formula. There is a possibility that the reaction search unit 13 selects a plurality of reactant combinations for the reaction formula, and in this case, the reaction search unit 13 identifies the product for each of the plurality of reactant combinations. Alternatively, the reaction search unit 13 may not select any reactant combination compatible with the reaction formula, and in this case, the reaction search unit 13 does not identify a product.

As shown in step S108, the subsequent processing is changed in accordance with whether there is a reactant combination compatible with the i-th reaction formula.

In a case where one or more reactant combinations are selected (in step S108, YES), the processing proceeds to step S109. In step S109, the reaction search unit 13 stores the i-th reaction formula, and the reactant combination and the product corresponding to that reaction formula, for the subsequent processing for the reaction formula list that is currently selected.

On the other hand, in a case where no reactant combination is selected (in step S108, NO), the processing proceeds to step S110. In step S110, the reaction search unit 13 discards the i-th reaction formula. This processing indicates that that reaction formula is not considered in the reaction formula list that is currently selected.

As shown in step S111, the reaction search unit 13 processes all the reaction formulae of the reaction formula list that is currently selected. In a case where there is an unprocessed reaction formula (in step S111, NO), the processing proceeds to step S112. In step S112, the reaction search unit 13 increments the variable i by 1. This indicates that the reaction search unit 13 processes the next reaction formula.

After step S112, the processing returns to step S105, and the reaction search unit 13 executes steps S105 to S111 for the next reaction formula. In step S105, the reaction search unit 13 selects the i-th reaction formula, i.e., the next reaction formula. In step S106, the reaction search unit 13 sets the reactant candidates for the reaction formula. For the second and subsequent reaction formulae, the reaction search unit 13 sets a sum set of products obtained by the previous processing and reactant lists, as reactant candidates for the i-th reaction formula. In addition, the reaction search unit 13 identifies the products finally obtained up to the current stage in the reaction formula list that is currently selected, in other words, the products finally stored in step S109 up to the (i−1)-th reaction formula, as a latest product. The reason for identifying the latest product is to search a continuous reaction indicated by at least two reaction formulae. In step S107, the reaction search unit 13 selects a reactant combination compatible with the i-th reaction formula from the reactant candidates. In a case of searching the second and subsequent reaction formulae for realizing the continuous reaction, the reaction search unit 13 sets a constraint condition that at least one reactant of the reactant combination is the latest product to select a reactant combination compatible with the i-th reaction formula. The reaction search unit 13 identifies a product to be obtained from the reactant combination by the reaction formula. After that, the reaction search unit 13 executes steps S108 to S111.

In a case where all the reaction formulae of the reaction formula list that is currently selected are processed (in step S111, YES), the processing proceeds to step S113.

As shown in step S113, the reaction search unit 13 processes all the reaction formula lists. In a case where there is an unprocessed reaction formula list (in step S113, NO), the processing returns to step S103. The reaction search unit 13 selects a next reaction formula list in step S103, and executes steps S104 to S113 for the reaction formula list.

In a case where all the reaction formula lists are processed (in step S113, YES), the processing proceeds to step S114. In step S114, the reaction search unit 13 outputs a search result. For example, the reaction search unit 13 outputs at least one of the final products to be obtained based on the reactants in the reactant list and the chemical reaction for obtaining the final products, as the search result. The chemical reaction from which the final product is obtained may be a single reaction or a continuous reaction. A method for outputting the search result is not limited. For example, the reaction search unit 13 may store the search result in a given database, transmit the search result to another computer or computer system, or display the search result on a display device. Alternatively, the reaction search unit 13 may output the search result to another function module, for subsequent processing in the information processing system 10.

As described above, the reactant data may indicate an available reagent as the reactant, and the reaction formula data may indicate a reaction formula selected based on at least one of realizability, handleability, and a yield. In this case, the search result may be expected to indicate a final product or a chemical reaction that is relatively easily realized.

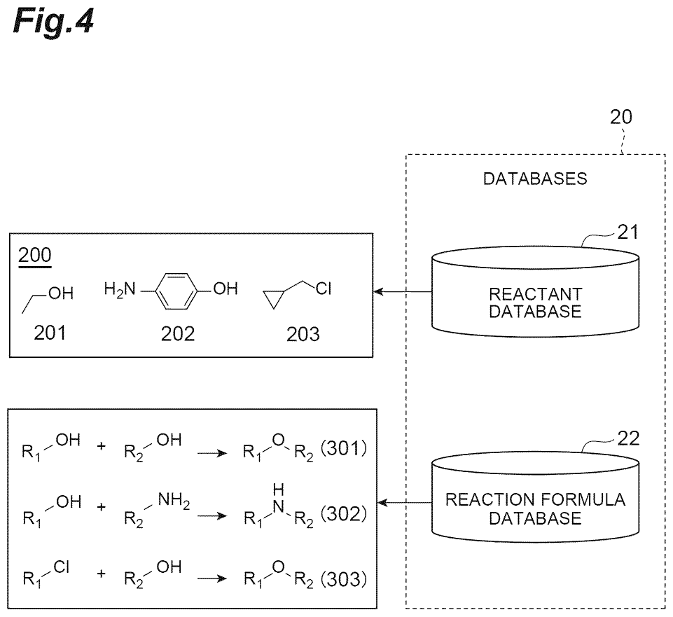

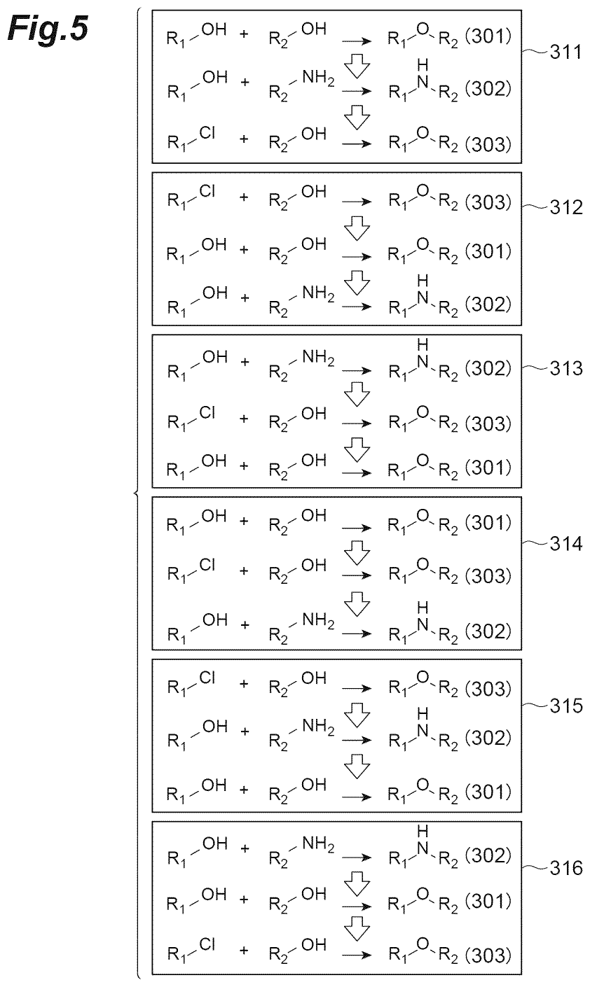

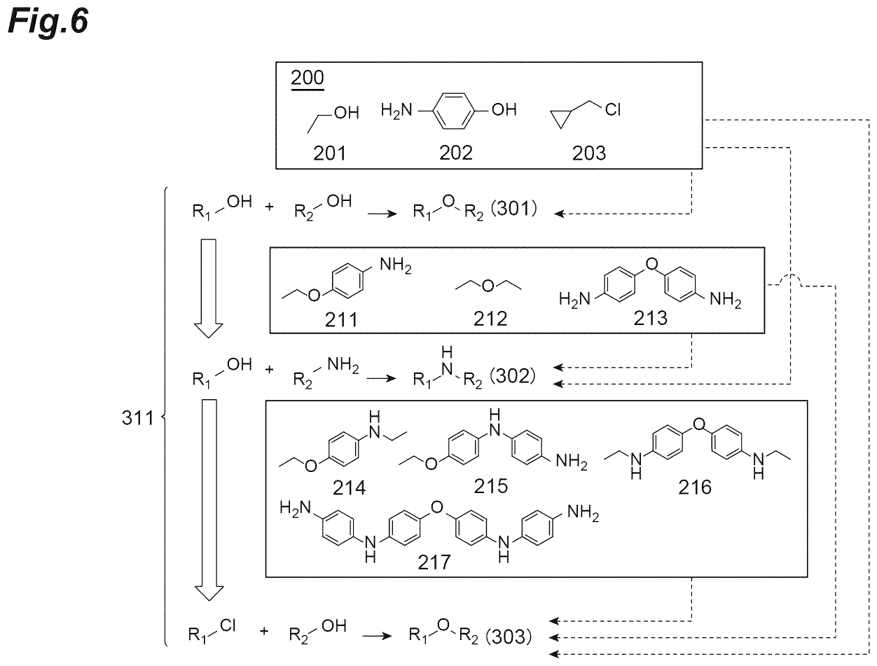

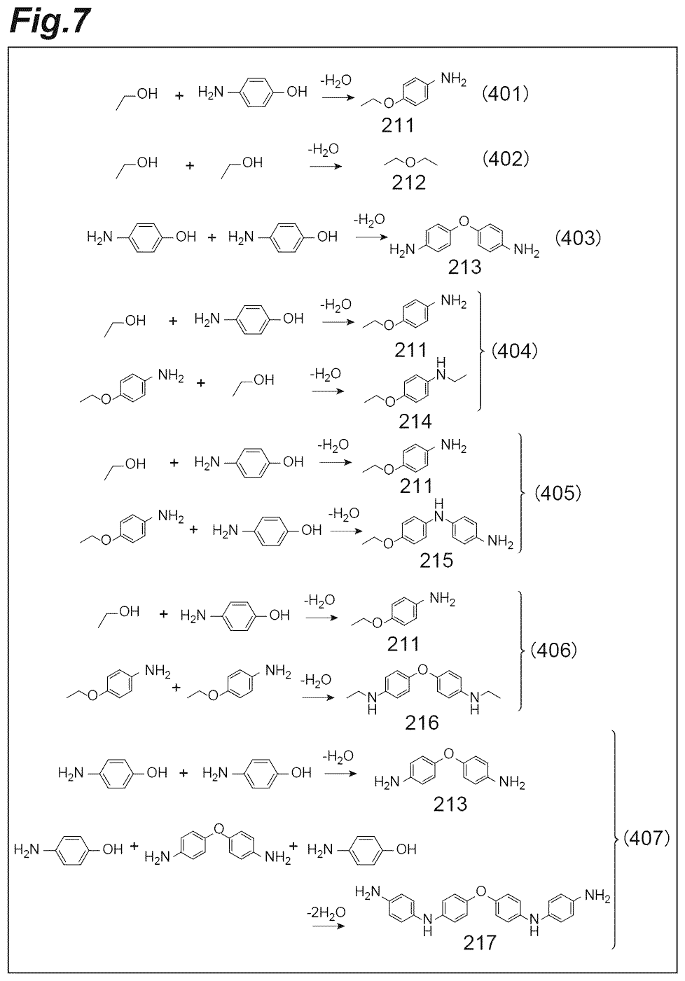

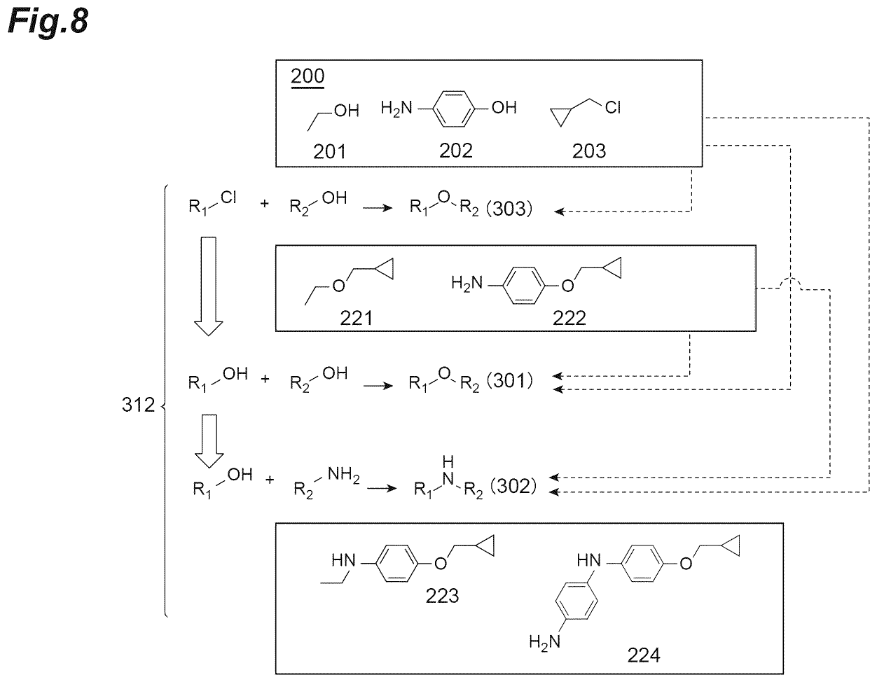

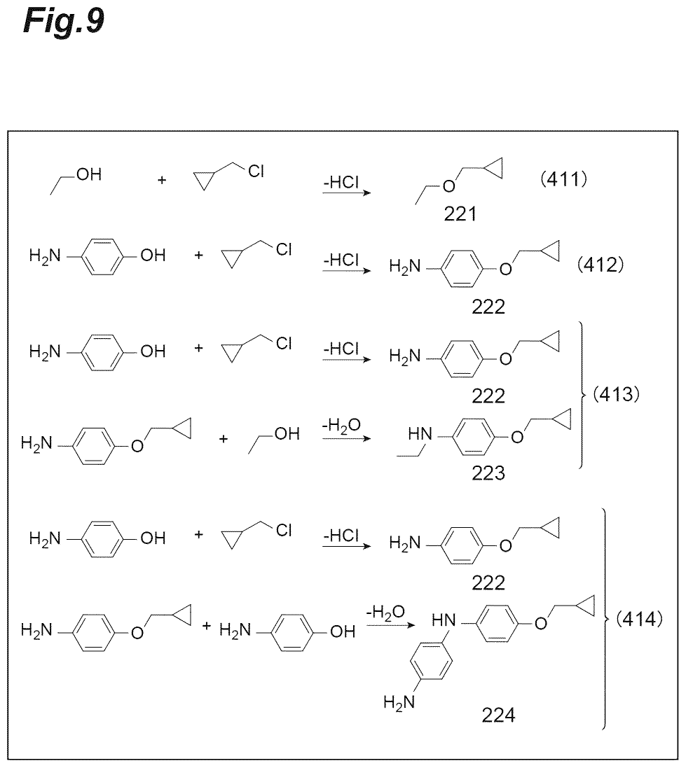

A specific example relevant to the processing flow S1 will be described with reference to FIG. 4 to FIG. 9. FIG. 4 is a diagram illustrating an example of reactants and reaction formulae acquired from the databases 20. FIG. 5 is a diagram illustrating an example of the reaction formula lists. FIG. 6 is a diagram illustrating an example of processing for one reaction formula list, and FIG. 7 is a diagram illustrating chemical reactions and products to be obtained by that processing. FIG. 8 is a diagram illustrating an example of processing for another reaction formula list, and FIG. 9 is a diagram illustrating chemical reactions and products to be obtained by that processing.

In step S101, as illustrated in FIG. 4, the reactant acquisition unit 11 reads out reactants 201 to 203 from the reactant database 21, and generates a reactant list 200 including these reactants. The reactant 201 is ethanol. The reactant 202 is p-aminophenol, and is also referred to as 4-hydroxyaniline. The reactant 203 is (chloromethyl) cyclopropane.

In step S102, as illustrated in FIG. 4, the reaction formula acquisition unit 12 reads out reaction formulae 301 to 303 from the reaction formula database 22. As illustrated in FIG. 5, the reaction formula acquisition unit 12 generates each of a plurality of arrangement patterns for those reaction formulae, as the reaction formula list. The reactive functional groups of two reactants indicated by the reaction formula 301 are both a hydroxy group. In the reaction formula 302, the reactive functional groups are a hydroxy group and an amino group. In the reaction formula 303, the reactive functional groups are a chloro group and a hydroxy group. The reaction formula acquisition unit 12 generates six reaction formula lists 311 to 316 by using the reaction formulae 301 to 303.

In step S103 and subsequent steps, the reaction search unit 13 searches a compound based on the reactant list 200 and the reaction formula lists 311 to 316.

In step S103 to S105, the reaction search unit 13 selects the reaction formula list 311, and selects the reaction formula 301 that is the first on the list.

In step S106, for the reaction formula 301, the reaction search unit 13 sets the entire reactant list 200 as reactant candidates.

In step S107, the reaction search unit 13 searches a reactant combination and a product compatible with the reaction formula 301. Hereinafter, a reactant combination consisting of two reactants R₁ and R₂ is represented as “{Reactant R₁, Reactant R₂}”, and a product to be obtained from the reactant combination is represented as “{Reactant R₁, Reactant R₂}->(Product)”. As illustrated in FIG. 6, the reaction search unit 13 selects {Reactant 201, Reactant 202}, {Reactant 201, Reactant 201}, and {Reactant 202, Reactant 202}, as reactant combinations compatible with the reaction formula 301. Then, the reaction search unit 13 identifies products 211 to 213 as follows.
{Reactant 201, Reactant 202}->(Product 211)
{Reactant 201, Reactant 201}->(Product 212)
{Reactant 202, Reactant 202}->(Product 213)

Since there are reactant combinations compatible with the reaction formula 301, the processing proceeds to step S109 after step S108. In step S109, the reaction search unit 13 stores the reaction formula 301, and three reactant combinations and three products 211 to 213 obtained in step S107.

In steps S111, S112, and S105, the reaction search unit 13 selects the reaction formula 302 that is the second on the reaction formula list 311.

In step S106, for the reaction formula 302, the reaction search unit 13 sets a sum set of the products 211 to 213 and the reactant list 200 as reactant candidates. In addition, the reaction search unit 13 identifies the finally obtained products 211 to 213 as latest products.

In step S107, the reaction search unit 13 searches a reactant combination and a product compatible with the reaction formula 302. The reaction search unit 13 selects a reactant combination compatible with the reaction formula 302 under a constraint condition that at least one of the reactant combination is any of the products 211 to 213. As illustrated in FIG. 6, the reaction search unit 13 selects {Reactant 211, Reactant 201}, {Reactant 211, Reactant 202}, {Reactant 213, Reactant 201}, and {Reactant 213, Reactant 202}, as reactant combinations compatible with the reaction formula 302. Then, the reaction search unit 13 identifies products 214 to 217 as follows. The product 216 is obtained from one reactant 213 and two reactants 201. The product 217 is obtained from one reactant 213 and two reactants 202.
{Reactant 211, Reactant 201}->(Product 214)
{Reactant 211, Reactant 202}->(Product 215)
{Reactant 213, Reactant 201}->(Product 216)
{Reactant 213, Reactant 202}->(Product 217)

Since there are reactant combinations compatible with the reaction formula 302, the processing proceeds to step S109 after step S108. In step S109, the reaction search unit 13 stores the reaction formula 302, and four reactant combinations and four products 214 to 217 obtained in step S107.

In steps S111, S112, and S105, the reaction search unit 13 selects the reaction formula 303 that is the third on the reaction formula list 311.

In step S106, for the reaction formula 303, the reaction search unit 13 sets a sum set of the products 211 to 217 obtained by the previous processing and the reactant list 200 as reactant candidates. In addition, the reaction search unit 13 identifies the finally obtained products 214 to 217 as latest products.

In step S107, the reaction search unit 13 searches a reactant combination and a product compatible with the reaction formula 303. The reaction search unit 13 selects a reactant combination compatible with the reaction formula 303 under a constraint condition that at least one of the reactant combination is any of the products 214 to 217. As illustrated in FIG. 6, since none of the products 214 to 217 has the reactive functional group indicated by the reaction formula 303, the reaction search unit 13 does not select any reactant combination compatible with the reaction formula 303.

Since there is no reactant combination compatible with the reaction formula 303, the processing proceeds to step S110 after step S108. In step S110, the reaction search unit 13 discards the reaction formula 303.

As illustrated in FIG. 7, the reaction search unit 13 identifies the products 211 to 217 as final products, based on the reaction formula list 311. The reaction search unit 13 may further identify chemical reactions 401 to 407 for obtaining the products 211 to 217. The chemical reactions 404 to 407 are continuous reactions.

Since all the reaction formulae of the reaction formula list 311 are processed, the processing proceeds to step S113 after step S111. Since there is an unprocessed reaction formula list, the processing returns to 103.

In steps S103 to S105, the reaction search unit 13 selects the reaction formula list 312, and selects the reaction formula 303 that is the first on the list.

In step S106, for the reaction formula 303, the reaction search unit 13 sets the entire reactant list 200 as reactant candidates.

In step S107, the reaction search unit 13 searches a reactant combination and a product compatible with the reaction formula 303. As illustrated in FIG. 8, the reaction search unit 13 selects {Reactant 201, Reactant 203} and {Reactant 202, Reactant 203}, as reactant combinations compatible with the reaction formula 303. Then, the reaction search unit 13 identifies products 221 and 222 as follows.
{Reactant 201, Reactant 203}->(Product 221)
{Reactant 202, Reactant 203}->(Product 222)

Since there are reactant combinations compatible with the reaction formula 303, the processing proceeds to step S109 after step S108. In step S109, the reaction search unit 13 stores the reaction formula 303, and two reactant combinations and two products 221 and 222 obtained in step S107.

In steps S111, S112, and S105, the reaction search unit 13 selects the reaction formula 301 that is the second on the reaction formula list 312.

In step S106, for the reaction formula 301, the reaction search unit 13 sets a sum set of the products 221 and 222 and the reactant list 200 as reactant candidates. In addition, the reaction search unit 13 identifies the finally obtained products 221 and 222 as latest products.

In step S107, the reaction search unit 13 searches a reactant combination and a product compatible with the reaction formula 301. The reaction search unit 13 selects a reactant combination compatible with the reaction formula 301 under a constraint condition that at least one of the reactant combination is any of the products 221 and 222. As illustrated in FIG. 8, since none of the products 221 and 222 has the reactive functional group indicated by the reaction formula 301, the reaction search unit 13 does not select any reactant combination compatible with the reaction formula 301.

Since there is no reactant combination compatible with the reaction formula 301, the processing proceeds to step S110 after step S108. In step S110, the reaction search unit 13 discards the reaction formula 301.

In steps S111, S112, and S105, the reaction search unit 13 selects the reaction formula 302 that is the third on the reaction formula list 312.

In step S106, for the reaction formula 302, the reaction search unit 13 sets a sum set of the products 221 and 222 obtained by the previous processing and the reactant list 200 as reactant candidates. In addition, the reaction search unit 13 identifies the finally obtained products 221 and 222 as latest products.

In step S107, the reaction search unit 13 searches a reactant combination and a product compatible with the reaction formula 302. The reaction search unit 13 selects a reactant combination compatible with the reaction formula 302 under a constraint condition that at least one of the reactant combination is any of the products 221 and 222. As illustrated in FIG. 8, the reaction search unit 13 selects {Reactant 222, Reactant 201} and {Reactant 222, Reactant 202}, as reactant combinations compatible with the reaction formula 302. Further, the reaction search unit 13 identifies products 223 and 224 obtained from the reactant combinations by the reaction formula 302.
{Reactant 222, Reactant 201}->(Product 223)
{Reactant 222, Reactant 202}->(Product 224)

Since there are reactant combinations compatible with the reaction formula 302, the processing proceeds to step S109 after step S108. In step S109, the reaction search unit 13 stores the reaction formula 302, and two reactant combinations and two products 223 and 224 obtained in step S107.

As illustrated in FIG. 9, the reaction search unit 13 identifies the products 221 to 224 as final products, based on the reaction formula list 312. The reaction search unit 13 may further identify chemical reactions 411 to 414 for obtaining the products 221 to 224. The chemical reactions 413 and 414 are continuous reactions.

Since all the reaction formulae of the reaction formula list 312 are processed, the processing proceeds to step S113 after step S111. After that, the reaction search unit 13 searches a compound for each of the reaction formula lists 313 to 316.

In a case where the reaction formula lists 311 to 316 are processed, in step S114, the reaction search unit 13 outputs a search result. For example, the reaction search unit 13 outputs a search result indicating the products 211 to 217 and 221 to 224, as well as the chemical reactions 401 to 407 and 411 to 414. As a result of processing the reaction formula lists 311 to 316, there is a possibility that the same final products and the same chemical reactions are obtained in two or more reaction formula lists. In this case, the reaction search unit 13 outputs the search result in a format excluding such overlaps.

In the example of FIG. 6 to FIG. 9, the reaction search unit 13 searches, for each of the reaction formula lists 311 to 316, a final product to be obtained by a chemical reaction indicated using at least one reaction formula, based on an arrangement order of a plurality of reaction formulae indicated by the reaction formula list and the reactant list 200. In this search, the reaction search unit 13 selects at least one reactant combination compatible with the reaction formula from the reactant list, and for each of the at least one reactant combination, identifies a product to be obtained from the reactant combination by the reaction formula. The reaction search unit 13 identifies the searched products 211 to 217 and 221 to 224 as final products. The reaction search unit 13 may further identify the chemical reactions 401 to 407 and 411 to 414. The final product to be searched may include a final product to be obtained by a continuous reaction indicated using at least two reaction formulae according to the arrangement order. Therefore, the chemical reaction to be identified may include the continuous reaction.

### [Use of Search Result]

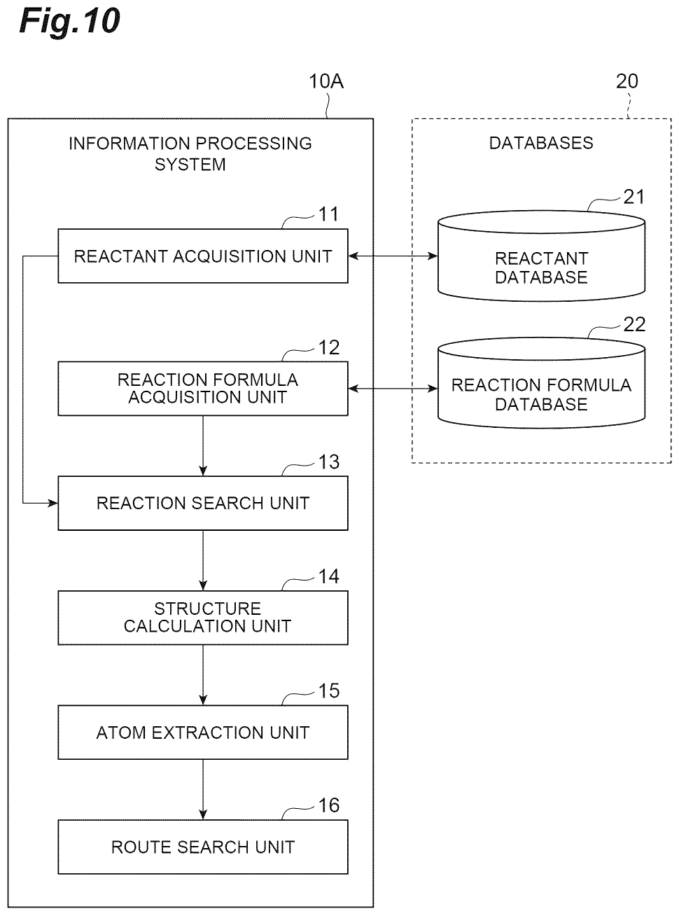

As described above, the information processing system according to the present disclosure may execute further processing on the obtained search result. FIG. 10 is a diagram illustrating an example of a functional configuration of an information processing system 10A executing that additional processing. The information processing system 10A is a computer system that searches a synthesizable compound and estimates a reaction route for each of one or more chemical reactions obtained as the search result. The reaction route refers to a process from a reactant to a product.

In an example, the information processing system 10A estimates the reaction route by using the Nudged Elastic Band (NEB) method. In the NEB method, the structure of a reactant and a product, that is, an initial structure and a final structure are processed as input data, and n intermediate structures connecting these two structures is generated. The intermediate structure is also referred to as an image. Each of the intermediate structures is bonded to another adjacent structure by a spring along the reaction route. The NEB method obtains a force exerted on each of the intermediate structures, and executes structure optimization while considering a vertical component with respect to the reaction route and a restitution force due to the spring, in order to obtain a reaction route with the minimum activation energy as a most stable route.

The NEB method is capable of accurately searching the reaction route with the minimum activation energy, but has a disadvantage that it takes time for that search. The information processing system 10A is capable of more rapidly estimating a reaction route from a plurality of reactants to a product.

The information processing system 10A includes a reactant acquisition unit 11, a reaction formula acquisition unit 12, a reaction search unit 13, a structure calculation unit 14, an atom extraction unit 15, and a route search unit 16, as function modules. That is, the information processing system 10A is realized by adding the structure calculation unit 14, the atom extraction unit 15, and the route search unit 16 to the information processing system 10. The structure calculation unit 14 is a function module calculating an optimum structure of each of a plurality of reactants and a product. The optimum structure refers to a structure of a substance in a case where the substance is in the minimum energy state, and is also referred to as a most stable structure. The atom extraction unit 15 is a function module extracting an atom relevant to the chemical reaction as a target atom, for each of the plurality of reactants. The route search unit 16 is a function module estimating a reaction route from the plurality of reactants to the product by at least the NEB method. The route search unit 16 sets a constraint condition of the NEB method limited to the target atom, and executes the NEB method under the constraint condition to estimate the reaction route.

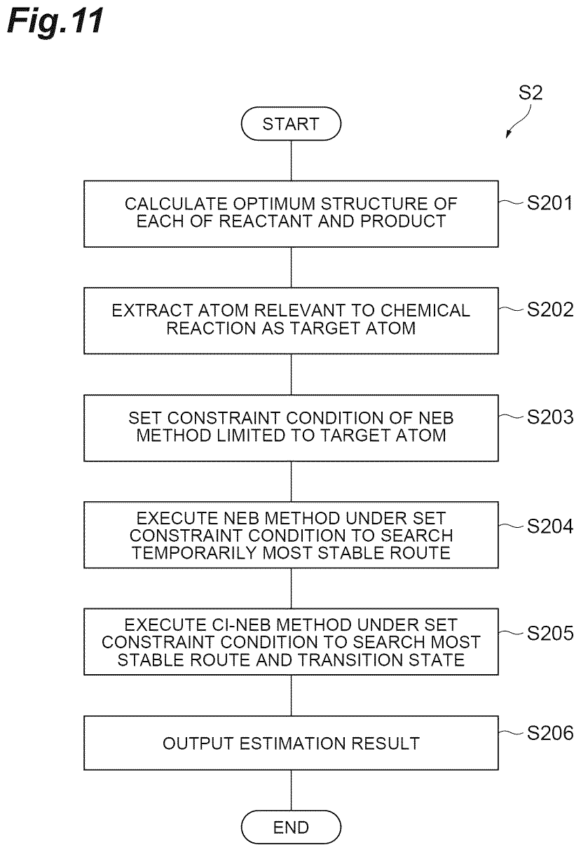

With reference to FIG. 11, the estimation of the reaction route by the information processing system 10A will be described. FIG. 11 is a flowchart illustrating an example of the estimation as a processing flow S2. In an example, the processing flow S2 is executed for each chemical reaction obtained by the processing flow S1.

In step S201, the structure calculation unit 14 calculates an optimum structure of each of a plurality of reactants and a product. In response to the reaction search unit 13 identifying at least one product, the structure calculation unit 14 receives data indicating the reactants and the product, and calculates an optimum structure of each substance by first-principles calculation. The first-principles calculation refers to a method for calculating physical properties of a substance based on quantum mechanics, without using empirical parameters, i.e., experiment data. A specific method for the first-principles calculation is not limited. In an example, the structure calculation unit 14 may be implemented using software for computational chemistry “Gaussian16”, manufactured by Gaussian, Inc., and the optimum structure may be calculated by a calculation condition such as B3LYP/6-31G(d).

In step S202, the atom extraction unit 15 extracts an atom relevant to the chemical reaction as a target atom. The “atom relevant to the chemical reaction” is an atom configuring a partial structure of a reactant changed in accordance with the chemical reaction (in other words, a reactant involved in a reaction route). The “atom relevant to the chemical reaction” may be rephrased to an “atom involved in the reaction route”. Therefore, for each of the reactants, the atom extracted as the target atom is only some atoms among all the atoms of the reactant. A method for extracting the atom relevant to the chemical reaction is not limited, and the atom extraction unit 15 may extract the target atom by any method.

In an example, the atom extraction unit 15 may select an atom in which an atomic distance is changed to cross a given threshold Ta due to a chemical reaction, and extract that atom as the target atom. There are two types of “atoms in which the atomic distance is changed to cross the threshold (Ta)”. One is an atom in which a distance with respect to the other atom is changed from a value greater than the threshold Ta to a value less than the threshold Ta due to a chemical reaction. Such a decrease in the atomic distance indicates that a bond is generated. More specifically, such a bond is a covalent bond. The other one is an atom in which a distance with respect to the other atom is changed from a value less than the threshold Ta to a value greater than the threshold Ta due to a chemical reaction. Such an increase in the atomic distance indicates that a bond (a covalent bond) is cleaved. That is, the atom in which the atomic distance is changed to cross the threshold is an atom relevant to the generation or the cleavage of the bond. The “atom relevant to the generation or the cleavage of the bond” is an example of the atom relevant to the chemical reaction.

In an example, the threshold Ta for a certain combination of two atoms is the product of a bond distance d of the combination and a coefficient α greater than 1. That is, Ta=d×α. The bond distance refers to an average distance between two atoms configuring a covalent bond, and more specifically, refers to an average distance between two atomic nuclei. The bond distance is determined by the combination of two atoms. The coefficient α is a common value that does not depend on the type of combination of two atoms. Therefore, the threshold Ta depends on the bond distance d. The common coefficient α may be determined by any policy, and for example, may be 1.2.

The atom extraction unit 15 extracts an atom in which the atomic distance is changed to cross the threshold Ta as the target atom, with reference to the optimum structure of each of the plurality of reactants and the product.

In another example, the atom extraction unit 15 may select an atom in which at least one bond angle is changed to cross a given threshold Tb due to a chemical reaction, and extract this atom as the target atom. The bond angle refers to an angle formed between two chemical bonds extending from atoms. There are two types of “atoms in which at least one bond angle is changed to cross the threshold (Tb)”. One is an atom in which an angle formed between two chemical bonds extending from atoms is changed from a value greater than the threshold Tb to a value less than the threshold Tb due to a chemical reaction. The other one is an atom in which an angle formed between two chemical bonds extending from atoms is changed from a value less than the threshold Tb and to a value greater than the threshold Tb due to a chemical reaction. There is a possibility that the atom in which the bond angle is changed to cross the threshold Tb is also involved in the reaction route.

In another example, the atom extraction unit 15 may select an atom group in which a dihedral angle is changed by a given threshold Tc or more due to a chemical reaction, and extract that atom group as the target atoms. The “atom group in which the dihedral angle is changed by the threshold (Tc) or more” may be two atoms common to two surfaces forming the dihedral angle, and an atom bonded to each of the two atoms. There is a possibility that the atom group in which the dihedral angle is changed by the threshold Tc or more is also involved in the reaction route.

In the present disclosure, the atom in which the atomic distance is changed to cross the threshold Ta, the atom in which the bond angle is changed to cross the threshold Tb, and the atom group in which the dihedral angle is changed by the threshold Tc or more are also referred to as a “first atom”. The atom extraction unit 15 may also extract an atom at a position close to the first atom in a reactant, as the target atom. In the present disclosure, such an atom is referred to as a “second atom”. Since the second atom is close to a position where the generation or the cleavage of the bond occurs or a position where there is a possibility that the generation or the cleavage of the bond occurs, the second atom may also be an atom relevant to the chemical reaction. In an example, the atom extraction unit 15 selects an atom bonded to the first atom in the reactant as the second atom, and extracts the second atom as the target atom.

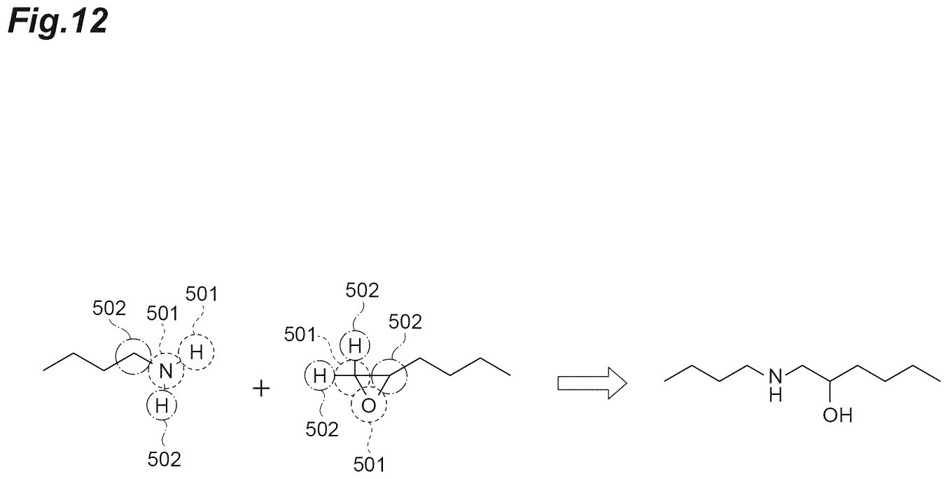

FIG. 12 is a diagram illustrating an example of extracting the target atom. This example indicates the extraction of the target atom in a reaction between an amine compound and an epoxy compound. This example indicates n-butyl amine as the amine compound, and indicates 1,2-epoxy hexane as the epoxy compound. In this example, the atom extraction unit 15 selects an atom in which the atomic distance is changed to cross the given threshold Ta due to the chemical reaction, as a first atom 501. Specifically, the atom extraction unit 15 selects a nitrogen atom and one hydrogen atom of the n-butyl amine, and an oxygen atom and a carbon atom on the front end of the 1,2-epoxy hexane, as the first atoms 501. Further, the atom extraction unit 15 selects an atom bonded to the first atom 501 as a second atom 502. Specifically, for the n-butyl amine, the atom extraction unit 15 selects another hydrogen atom and one carbon atom, each of which is bonded to the nitrogen atom, as the second atoms 502. In addition, for the 1,2-epoxy hexane, the atom extraction unit 15 selects a carbon atom bonded to both of the oxygen atom and the carbon atom on the front end, and two hydrogen atoms bonded to the carbon atom on the front end, as the second atoms 502. Therefore, the atom extraction unit 15 extracts a total of nine atoms as the target atoms.

Returning to FIG. 11, in step S203, the route search unit 16 sets a constraint condition of the NEB method limited to each of the target atoms. That is, the route search unit 16 sets the constraint condition limited to some atoms for each of the reactants. Specifically, the route search unit 16 sets a constraint condition that a reaction direction of each target atom is limited to the direction of the reaction route. This constraint condition includes a restriction that each target atom is bonded to the adjacent intermediate structure by a spring along the reaction route. A spring constant included in the constraint condition may be set based on any policy.

In step S204, the route search unit 16 executes the NEB method under the set constraint condition to search a temporarily most stable route. Specifically, the route search unit 16 generates a certain reaction route indicating a change in the coordinates of each atom between the reactant and the product. The reaction route may be referred to as an initial reaction route. Subsequently, the route search unit 16 sets a plurality of intermediate structures on the reaction route. Each intermediate structure may be referred to as a waypoint on the reaction route. Subsequently, for each of the plurality of intermediate structures, the route search unit 16 calculates a potential energy of the intermediate structure and a force that is the first derivation of the potential energy. Then, the route search unit 16 executes the structure optimization of each intermediate structure based on the calculation result to update the coordinates of each atom of each intermediate structure. As a result, a new reaction route is obtained. The route search unit 16 executes a series of processes including the calculation of the potential energy and the force, the structure optimization, and the update of the coordinates of each atom, under the constraint condition. The route search unit 16 repeatedly executes the series of processes until the change amount of the potential energy is a given threshold or less in each intermediate structure. In a case where the change amount of the potential energy is the given threshold or less for the finally obtained reaction route, the route search unit 16 ends the repetition processing, and estimates the route as the temporarily most stable route.

In this embodiment, the route search unit 16 calculates a transition state (TS) in a chemical reaction from the route obtained by the NEB method and the most stable route via the transition state, by using a CI-NEB method. Therefore, in this embodiment, the route obtained by the NEB method is expressed as the “temporarily most stable route”. The transition state refers to a state with the highest energy in a chemical reaction. The CI-NEB method is the improved method of the NEB method.

In step S205, the route search unit 16 executes the CI-NEB method in the set constraint condition to search the most stable route and the transition state. Specifically, the route search unit 16 reads in data indicating the temporarily most stable route obtained by the NEB method. Subsequently, for each of the plurality of intermediate structures, the route search unit 16 calculates the potential energy of the intermediate structure and the force that is the first derivation of the potential energy. In such calculation, the route search unit 16 considers a force for the intermediate structure to climb a potential surface, without introducing the concept of a spring, with respect to the intermediate structure (the image) with the highest energy. For other intermediate structures, the route search unit 16 executes the same calculation as the NEB method. As described above, a difference between the CI-NEB method and the NEB method is a calculation method for the intermediate structure with the highest energy. The route search unit 16 executes the structure optimization of each intermediate structure based on the calculation result to update the coordinates of each atom of each intermediate structure. As a result, a new reaction route is obtained. The route search unit 16 executes a series of processes including the calculation of the potential energy and the force, the structure optimization, and the update of the coordinates of each atom, under the constraint condition. The route search unit 16 repeatedly executes the series of processes until the change amount of the potential energy is a given threshold or less in each intermediate structure. In a case where the change amount of the potential energy is the given threshold or less for the finally obtained reaction route, the route search unit 16 ends the repetition processing, and estimates the route as the most stable route.

In step S206, the route search unit 16 outputs the most stable route as an estimation result. A method for outputting the estimation result is not limited. For example, the route search unit 16 may store the estimation result in a given database, transmit the estimation result to another computer or computer system, or display the estimation result on a display device. Alternatively, the route search unit 16 may output the estimation result to another function module for the subsequent processing in the information processing system 10A.

In the processing flow S2, the route search unit 16 estimates the reaction route by using the NEB method and the CI-NEB method. However, the information processing system 10A may estimate the reaction route by the NEB method without using the CI-NEB method. For example, the information processing system 10A may output the “temporarily most stable route” estimated in step S204 as the final estimation result.

### Effect

As described above, the information processing system according to one aspect of the present disclosure includes at least one processor. At least one processor is configured to: acquire a reactant list indicating a plurality of reactants; acquire a reaction formula expressing a reactant having a reactive functional group by a general formula; select at least one combination of the reactants compatible with the reaction formula as at least one reactant combination from the reactant list; and identify, for each of the at least one reactant combination, a product to be obtained from the reactant combination by the reaction formula.

The information processing method according to one aspect of the present disclosure is executed by the information processing system including at least one processor. This information processing method includes: acquiring a reactant list indicating a plurality of reactants; acquiring a reaction formula expressing a reactant having a reactive functional group by a general formula; selecting at least one combination of the reactants compatible with the reaction formula as at least one reactant combination from the reactant list; and identifying, for each of the at least one reactant combination, a product to be obtained from the reactant combination by the reaction formula.

The information processing program according to one aspect of the present disclosure causes the computer to execute: acquiring a reactant list indicating a plurality of reactants; acquiring a reaction formula expressing a reactant having a reactive functional group by a general formula; selecting at least one combination of the reactants compatible with the reaction formula as at least one reactant combination from the reactant list; and identifying, for each of the at least one reactant combination, a product to be obtained from the reactant combination by the reaction formula.

In such aspects, by selecting the reactant combination and identifying the product based on the reaction formula focusing on the reactive functional group that directly contributes to the chemical reaction, it is possible to efficiently search the synthesizable compound.

In the information processing system according to another aspect, at least one processor may be configured to: acquire a plurality of the reaction formulae; generate each of a plurality of arrangement patterns for the plurality of reaction formulae as a reaction formula list; search, for each of a plurality of the reaction formula lists, a final product to be obtained by a chemical reaction indicated using at least one of the reaction formulae, based on an arrangement order of the plurality of reaction formulae indicated by the reaction formula list and the reactant list; and identify the searched final product. By using such a reaction formula list, it is possible to comprehensively identify the final product that may be generated.

In the information processing system according to another aspect, at least one processor may be configured to further identify the chemical reaction corresponding to the final product. By such processing, it is possible to obtain the chemical reaction for obtaining the final product.

In the information processing system according to another aspect, at least one processor may be configured to search, for each of the plurality of reaction formula lists, the final product to be obtained by a continuous reaction indicated using at least two of the reaction formulae according to the arrangement order. In this case, it is possible to identify the final product that may be generated by the continuous reaction.

In the information processing system according to another aspect, at least one processor may be configured to further identify the continuous reaction corresponding to the final product. By such processing, it is possible to obtain the continuous reaction for obtaining the final product.

In the information processing system according to another aspect, at least one processor may be configured to, for each of the plurality of reaction formula lists: in a case where there is the at least one reactant combination compatible with an i-th reaction formula, store the i-th reaction formula, the at least one reactant combination, and at least one of the product to be obtained from the at least one reactant combination by the i-th reaction formula; and under a constraint condition that at least one reactant of the reactant combination compatible with an (i+1)-th reaction formula is the stored product, search the at least one reactant combination compatible with the (i+1)-th reaction formula, in response to the stored product. The product to be obtained by the i-th reaction formula is temporarily set as the intermediate product, and the reactant combination compatible with the next reaction formula is searched under the constraint condition. By such processing, it is possible to comprehensively search the continuous reaction.

In the information processing system according to another aspect, at least one processor may be configured to, in response to the identified product: extract, for each of a plurality of reactants included in the reactant combination corresponding to the identified product, some atoms relevant to a chemical reaction indicated by the reaction formula as a target atom, among all atoms of the reactant; set a constraint condition of an NEB method limited to the target atom; and execute the NEB method under the constraint condition to estimate a reaction route from the plurality of reactants to the product. In this case, the constraint condition of the NEB method is set limited to some atoms relevant to the chemical reaction, among all the atoms of the reactant. Then, the reaction route is estimated by the NEB method under the constraint condition. Since a calculation time for the NEB method is shortened by limiting the atoms for which the constraint condition is set, that is, since the structure optimization more rapidly converges, it is possible to rapidly estimate the reaction route from the plurality of reactants to the product.

In the information processing system according to another aspect, at least one processor may be configured to select, for each of the plurality of reactants, an atom in which an atomic distance is changed to cross a given threshold due to the chemical reaction as a first atom, and extract the first atom as the target atom. A change in the atomic distance indicates the generation or the cleavage of the bond. Therefore, by considering the atomic distance, it is possible to appropriately extract the target atom for which the constraint condition is set. Accordingly, it is possible to estimate the reaction route with a higher accuracy.

In the information processing system according to another aspect, the threshold may be a product of a bond distance and a coefficient greater than 1. By setting the threshold in such a manner, it is possible to accurately select the atom relevant to the generation or the cleavage of the bond based on the atomic distance. As a result, it is possible to appropriately extract the target atom for which the constraint condition is set, and estimate the reaction route with a higher accuracy.

In the information processing system according to another aspect, at least one processor may be configured to select, for each of the plurality of reactants, an atom in which a bond angle is changed to cross a given threshold due to the chemical reaction as a first atom, and extract the first atom as the target atom. A change in the bond angle indicates that there is a possibility that the atom is involved in the reaction route. Therefore, by considering the bond angle, it is possible to appropriately extract the target atom for which the constraint condition is set. Accordingly, it is possible to estimate the reaction route with a higher accuracy.

In the information processing system according to another aspect, at least one processor may be configured to select, for each of the plurality of reactants, an atom group in which a dihedral angle is changed by a given threshold or more due to the chemical reaction as a first atom, and extract the first atom as the target atom. A change in the dihedral angle indicates that there is a possibility that the atom group is involved in the reaction route. Therefore, by considering the dihedral angle, it is possible to appropriately extract the target atom for which the constraint condition is set. Accordingly, it is possible to estimate the reaction route with a higher accuracy.

In the information processing system according to another aspect, the target atom may include a second atom bonded to the first atom. By setting not only the first atom relevant to the generation or the cleavage of the bond but also the second atom adjacent to the first atom as a target for setting the constraint condition, it is possible to estimate the reaction route with a higher accuracy.

### Modification Example

The detailed description has been made based on the embodiment of the present disclosure. However, the present disclosure is not limited to the examples described above. The present disclosure can be variously modified in a range not departing from the gist thereof.

The reaction search unit 13 may apply a given restriction condition to the final product obtained by steps S101 to S113 of the processing flow S1, and output the final product satisfying the restriction condition as the final result. This restriction condition may be that the final product may include given n molecular structures, or that the molecular weight of the final product may be a given threshold or more or a given threshold or less. Alternatively, the reaction search unit 13 may apply a given restriction condition to the reactant identified in step S107. In this example, in a case where there is the reactant combination compatible with the i-th reaction formula and the product satisfies the restriction condition, the reaction search unit 13 may execute step S109, otherwise, the reaction search unit 13 may execute step S110.

The information processing system may execute processing corresponding to the processing flow S2 by a learned model obtained by machine learning.

The processing procedure of the method to be executed by at least one processor is not limited to the examples in the embodiment described above. For example, part of the processing pieces or the steps described above may be omitted, or each of the steps may be executed in another order. In addition, any two or more steps among the steps described above may be combined, or part of the steps may be modified or deleted. Alternatively, other steps may be executed in addition to each of the steps described above.

In a case where comparing a magnitude relationship between two numerical values in the information processing system, either of two criteria such as “or more” and “greater than” may be used, or either of two criteria such as “or less” and “less than” may be used. The selection of such criteria does not change the technical significance for processing of comparing the magnitude relationship between two numerical values.

In the present disclosure, the expression “at least one processor executes the first processing, executes the second processing, . . . , and executes the n-th processing.” or an expression corresponding thereto indicates a concept including a case where a processor executing n processing pieces from the first processing to the n-th processing is changed in the middle. That is, that expression indicates a concept including both of a case where all of n processing pieces are executed by the same processor and a case where the processor is changed by a certain policy.

### REFERENCE SIGNS LIST

10, 10A: information processing system, 11: reactant acquisition unit, 12: reaction formula acquisition unit, 13: reaction search unit, 14: structure calculation unit, 15: atom extraction unit, 16: route search unit, 20: database group, 21: reactant database, 22: reaction formula database, 200: reactant list, 311 to 316: reaction formula lists.

1. An information processing system, comprising at least one processor,
wherein the at least one processor is configured to:
acquire a reactant list indicating a plurality of reactants;
acquire a reaction formula expressing a reactant having a reactive functional group by a general formula;
select at least one combination of the reactants compatible with the reaction formula as at least one reactant combination from the reactant list; and
identify, for each of the at least one reactant combination, a product to be obtained from the reactant combination by the reaction formula.

2. The information processing system according to claim 1,
wherein the at least one processor is configured to:
acquire a plurality of the reaction formulae;
generate each of a plurality of arrangement patterns for the plurality of reaction formulae as a reaction formula list;
search, for each of a plurality of the reaction formula lists, a final product to be obtained by a chemical reaction indicated using at least one of the reaction formulae, based on an arrangement order of the plurality of reaction formulae indicated by the reaction formula list and the reactant list; and
identify the searched final product.

3. The information processing system according to claim 2,
wherein the at least one processor is configured to further identify the chemical reaction corresponding to the final product.

4. The information processing system according to claim 2,
wherein the at least one processor is configured to search, for each of the plurality of reaction formula lists, the final product to be obtained by a continuous reaction indicated using at least two of the reaction formulae according to the arrangement order.

5. The information processing system according to claim 4,
wherein the at least one processor is configured to further identify the continuous reaction corresponding to the final product.

6. The information processing system according to claim 4,
wherein the at least one processor is configured to, for each of the plurality of reaction formula lists:
in a case where there is the at least one reactant combination compatible with an i-th reaction formula, store the i-th reaction formula, the at least one reactant combination, and at least one of the product to be obtained from the at least one reactant combination by the i-th reaction formula; and
under a constraint condition that at least one reactant of the reactant combination compatible with an (i+1)-th reaction formula is the stored product, search the at least one reactant combination compatible with the (i+1)-th reaction formula, in response to the stored product.

7. The information processing system according to claim 1,
wherein the at least one processor is configured to, in response to the identified product:
extract, for each of a plurality of reactants included in the reactant combination corresponding to the identified product, some atoms relevant to a chemical reaction indicated by the reaction formula as a target atom, among all atoms of the reactant;
set a constraint condition of an NEB method limited to the target atom; and
execute the NEB method under the constraint condition to estimate a reaction route from the plurality of reactants to the product.

8. An information processing method executed by an information processing system including at least one processor, the method comprising:
acquiring a reactant list indicating a plurality of reactants;
acquiring a reaction formula expressing a reactant having a reactive functional group by a general formula;
selecting at least one combination of the reactants compatible with the reaction formula as at least one reactant combination from the reactant list; and
identifying, for each of the at least one reactant combination, a product to be obtained from the reactant combination by the reaction formula.

9. A non-transitory computer-readable storage medium storing an information processing program for causing a computer to execute:
acquiring a reactant list indicating a plurality of reactants;
acquiring a reaction formula expressing a reactant having a reactive functional group by a general formula;
selecting at least one combination of the reactants compatible with the reaction formula as at least one reactant combination from the reactant list; and
identifying, for each of the at least one reactant combination, a product to be obtained from the reactant combination by the reaction formula.
